(19) United States
(12) Patent Application Publication
(10) Pub. No.: US 2022/0399086 A1
(43) Pub. Date: Dec. 15, 2022

(54) CLASSIFYING AND ANSWERING MEDICAL INQUIRIES BASED ON MACHINE-GENERATED DATA RESOURCES AND MACHINE LEARNING MODELS

(71) Applicant: Surescripts, LLC, Arlington, VA (US)

(72) Inventors: Bradley Carter Simons, Rosemount, MN (US); Robert Driskill, Greenfield, MN (US); Andrew Mellin, St. Paul, MN (US)

(21) Appl. No.: 17/342,973

(22) Filed: Jun. 9, 2021

(51) Int. Cl. G16H 10/20; G06F 16/36; G06F 16/33; G06F 16/35; G06N 20/00; G16H 10/60; G16H 50/20; G16H 30/20; G06F 40/30

(52) U.S. Cl. CPC G16H 10/20; G06F 16/367; G06F 16/3331; G06F 16/35; G06N 20/00; G16H 10/60; G16H 50/20; G16H 30/20; G06F 40/30

(57) ABSTRACT

Systems, methods, and devices are described for classifying and answering medical inquiries based on machine-generated data resources and machine learning models. A CCDA document including clinical information and observations of a patient are received from a requestor and utilized to generate a FHIR model instance specific to the CCDA document. Question text having medical inquires for the patient, and received with the CCDA document, is processed by a machine learning model to determine question categories for the medical inquiries which are utilized to map the inquires to objects of the model instance using another machine learning model. The model instance is queried based on the mapping to return values associated with the inquiries. The values are transmitted back to the requestor.

### BACKGROUND

Clinical Document Architecture (CDA) is an HL7 (“Health Level Seven International”) standard for building electronic clinical documents. Consolidated-CDA (C-CDA or CCDA) was subsequently developed to address shortcomings with the original CDA standard. CCDA document formats are a common way to exchange clinical information, in part due to the Meaningful Use Stage Two (MU2) criteria which call out use of C-CDA documents for particular types of information exchange.

Fast Healthcare Interoperability Resources (FHIR) is a recent standard for clinical messaging/interoperability in which healthcare information is conveyed electronically (see https://www.h17.org/fhir/overview.html). FHIR provides, at its base level, a certain degree of specificity for fundamental atoms of information in clinical messaging, thus providing a representation for clinical information that allows for interchange of information at levels more granular than an entire document. The basic FHIR standard allows for specific profiles of information to be used by different entities.

### BRIEF SUMMARY

Methods, processing systems, and apparatuses are described for classifying and answering medical inquiries based on machine-generated data resources and machine learning models, substantially as shown in and/or described herein in connection with at least one of the figures, as set forth more completely in the claims. A CCDA (Consolidated Clinical Document Architecture) document including clinical information and observations of a patient are received from a requestor and utilized to generate a FHIR (Fast Healthcare Interoperability Resources) model instance specific to the CCDA document. Question text having medical inquires for the patient, and received with the CCDA document, is processed by a machine learning model to determine question categories for the medical inquiries which are utilized to map the inquires to objects of the model instance using another machine learning model. The model instance is queried based on the mapping to return values associated with the inquiries. The values are then transmitted back to the requestor to complete the request.

### BRIEF DESCRIPTION OF THE DRAWINGS/FIGURES

The accompanying drawings, which are incorporated herein and form a part of the specification, illustrate embodiments and, together with the description, further serve to explain the principles of the embodiments and to enable a person skilled in the pertinent art to make and use the embodiments.

FIG. 1 shows a block diagram of a computer system that includes a host for classifying and answering medical inquiries based on machine-generated data resources and machine learning models, according to an example embodiment.

FIG. 2 shows a block diagram of a network of computer systems that includes a host for classifying and answering medical inquiries based on machine-generated data resources and machine learning models, according to an example embodiment.

FIG. 3 shows a block diagram of an inquiry classifier and response system for classifying and answering medical inquiries based on machine-generated data resources and machine learning models inquiry classifier and response system, according to an example embodiment.

FIG. 4 shows a flowchart of a method for includes a host for classifying and answering medical inquiries based on machine-generated data resources and machine learning models, according to an example embodiment.

FIG. 5 shows a system execution flow for classifying and answering medical inquiries based on machine-generated data resources and machine learning models, according to an example embodiment.

FIG. 6 shows a flowchart of a method for classifying and answering medical inquiries based on machine-generated data resources and machine learning models, according to an example embodiment.

FIG. 7 shows a flowchart of a method for classifying and answering medical inquiries based on machine-generated data resources and machine learning models, according to an example embodiment.

FIG. 8 shows a flowchart of a method for classifying and answering medical inquiries based on machine-generated data resources and machine learning models, according to an example embodiment.

FIG. 9 shows an ontology model, according to an example embodiment.

FIG. 10 shows a block diagram of a processing device/system in which the techniques disclosed herein may be performed and the embodiments herein may be utilized.

Embodiments will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION

### I. Introduction

The present specification discloses numerous example embodiments. The scope of the present patent application is not limited to the disclosed embodiments, but also encompasses combinations of the disclosed embodiments, as well as modifications to the disclosed embodiments.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Still further, it should be noted that the drawings/figures are not drawn to scale unless otherwise noted herein.

Numerous exemplary embodiments are now described. Any section/subsection headings provided herein are not intended to be limiting. Embodiments are described throughout this document, and any type of embodiment may be included under any section/subsection. Furthermore, it is contemplated that the disclosed embodiments may be combined with each other in any manner. That is, the embodiments described herein are not mutually exclusive of each other and may be practiced and/or implemented alone, or in any combination.

### II. Example Inquiry Classifier and Response System Embodiments

The example techniques and embodiments described herein utilize machine learning models for classifying and answering medical inquiries based on machine-generated data resources and machine learning (ML) models. An ML model that is trained on prior medical inquiries is utilized to determine categories for received medical inquiries, e.g., as text question data, from requestors. Requestors may include, without limitation, one or more of supervising physician organizations (SPOs)/prescribers, pharmacies/pharmacists, pharmacy benefits management (PBM) entities, initiators of electronic prior authorization (ePA) for prescription medications, insurance providers, and/or other types of health care providers or associated entities. Categories under which medical inquiries that can be answered are determined by an analysis of one or more clinical data sources (e.g., CCDA (Consolidated Clinical Document Architecture), FHIR (Fast Healthcare Interoperability Resources) queries, medication histories, fill data, and/or the like) of the patient. Embodiments herein also provide for another ML model that is trained on prior medical inquiries and/or answers to determine if a question is answerable by the systems and devices described herein prior to determining medical inquiry categories. Additionally, an ML category model is configured to determine answers for medical inquiries from the appropriate clinical data source based on the determined category(ies). These answers may be either directly sourced from a data source or may include generated data that is based on a data source (e.g., age of the patient can be generated based on identifying a patient birthdate).

Embodiments may be implemented as microservices in a computing system/host platform that service other software services/applications in which interactions by SPOs, ePA requestors, etc., are supplemented with the additional patient information determined by ML models and generated computing resources. For instance, any system, computing device process, application, and/or service for which determinations of data for patient-specific question and inquiries, e.g., in reporting, evaluation, implementing benefits, and/or question fulfillment, may utilize the embodiments herein. Many interactions between healthcare related entities involve determining answers to medical inquiries about a patient in order to perform actions, provide benefits, authorize/fulfill prescriptions, perform record keeping, and/or the like, which currently requires interactions with different data sources, extensive user interface (UI) interaction with requestor devices, additional overhead for processing/network resources in data source hosts that may be distributed, as well as interacting with data of different formats/standards, much less taking into account the inclusion of errors and inefficiencies introduced by members of healthcare related entities in gathering, collecting, and storing patients' medical information.

The described embodiments may be adapted to various types of systems and devices, for example but without limitation, computing systems (e.g., computers/computing devices such as desktops, laptops, etc., and servers, enterprise computing systems, etc.), communication devices (e.g., cellular and smart phones, etc.), and/or the like, that communicate information, such as in accordance with communication standards. For instance, computing systems that communicate over a network and exchange clinical information in accordance with the CCDA standard may be configured according to the described embodiments and techniques. While the embodiments herein may be described with respect to various computing systems and implementations as conceptual and/or illustrative examples for descriptive consistency, other types of electronic and communication devices and implementations are also contemplated for implementing the disclosed techniques. It is contemplated herein that in various embodiments and with respect to the illustrated figures of this disclosure, one or more components described and/or shown may not be included and that additional components may be included.

The example techniques and embodiments described herein provide for clinical resource generation using ontology models, e.g., such as FHIR resource generation, such as FHIR ontology model instances, using ontology models that map to information contained in CCDA documents, in processing systems, which is utilized, through ML classifications and mappings, to query the resources for information specific to medical inquiries. The processing systems may be a host entity such as a server or host server, as well as other server/client processing devices of requestors for clinical resource generation, as well as inquiry classification and responses, i.e., systems for classifying and answering medical inquiries based on machine-generated data resources and machine learning models, e.g., computing devices, of a trading partner(s), a vendor service(s), a doctor or doctor's office (including nurses and/or other staff), and/or another third-party entity(ies). The processing systems and/or users thereof may receive CCDA documents (e.g., in extensible markup language (XML) format) containing clinical information about various patients. The clinical information may include information like patient name, address, title, contact information, age, gender, clinical observations from doctor visits (e.g., weight, temperature, blood pressure, symptoms, diagnoses, and/or the like), prescriptions of the patient, etc. CCDA provides a structured identification system for clinical information using identifiers, e.g., tags, paired with information strings. For instance, an example patient name may be represented in a CCDA document as:

```
<patient>
  <name>
    <given>Paulina</given>
    <family>Coffin</family>
  </name>
  . . .
</patient>
```

Such identifiers and associated strings may be referred to herein as information-value pairs where the identifier tag describes of what type the information is and the value is the string. It should be noted that for a given type of information in an information-value pair, many values may be included in a CCDA for different people associated with the clinical data, such as, but without limitation, doctors, nurses, patients, legal guardians, etc.

The organizational structure of information in CCDA documents is not strictly defined, e.g., a patient name, such as the patient name noted above, may be included at different sections for different CCDA documents of various patients; an observation in one section of a CCDA document may be contained within organizer/composition components but in another section be contained in act/entryRelationship components and still in yet other sections the observation information may be in neither of these components. Hence, to be able to handle all the structures in a CCDA within which an observation may occur, FHIR ontology models are utilized in embodiments. In other words, names, dates, observations, and other information of interest to insurance providers, doctors/prescribers, pharmacists, pharmacy benefits management (PBM) entities, and/or other types of health care providers, may reside in various parts of any given CCDA document, and compounding this issue is the sheer volume of data that may be included in a given CCDA document, which essential, which may include tens or even hundreds of pages of clinical information without predictable organization, and therefore generating FHIR model instances with defined structures allows for a more efficient provision of specific data/information that responds to received medical inquiries.

The FHIR standard was promulgated to provide for communications having a certain degree of specificity for fundamental atoms of information in clinical messaging. That is, messaging between computer systems and querying databases that use FHIR includes the exchange and querying of clinical information that may relate to a number of defined “resources” associated with patients, practitioners, appointments, clinical observations, clinical documents, medications, accounts, and/or the like, as defined by the FHIR standard. For instance, FHIR patient resources may include information about a patient like patient name, address, title, contact information, age, gender, clinical observations from doctor visits (e.g., measured body weight, temperature, and blood pressure, symptoms, diagnoses, and/or the like), prescriptions of the patient, etc. A FHIR resource also utilizes libraries with objects that represent the resource. Generally, FHIR resources also include metadata (e.g., in formats such as, but without limitation, XML format) that describes different aspects of the resources. That is, the metadata may describe the structure and components of the resource (e.g., properties of the resource such as, but not limited to, a first property for a resource being an identification field, while a second property for the resource is a patient's last name, and a third property for the resource is a patient's first name, etc.), rather than actual informational values associated the resource (e.g., Resource ID=123456, Last name=“Berger”, and First name=“Devin”).

As described herein with respect to embodiments, clinical information may be exchanged between computer systems and/or queried against a database of FHIR models/resources according to the FHIR standard. Utilizing a standard Resource Description Framework (RDF) model, along with mapping information corresponding to a CCDA document, instances of FHIR models/resources for specific information from CCDA documents, including but not limited to clinical observations, can be automatically generated according to the described techniques and embodiments herein and utilized via ML models to generate answering communications for received medical inquiries.

As a non-limiting example, when a requestor provides a set of medical inquiries, e.g., as text question data, concerning a patient, the systems embodied herein are configured to take the questions and the CCDA of the patient and analyze the inquiries through an ML model(s) (e.g., based on a training set of prior inquiries for answerability) to determine if any inquiries are answerable from information in the CCDA. The CCDA is broken down into a collection of FHIR resources as noted above, and the text question data is submitted to a trained ML model (e.g., based on a training set of prior inquiries for categories) for classification into categories. The system queries for the relevant data from the FHIR resource objects in the FHIR model instance, i.e., the ontology model instance, via a category ML model that maps inquiry/category characteristics to the FHIR model instance, by applying applicable processes to generate new data as determined by the category. The query result may be either raw data or the generated data and is returned to the upstream system to be used to automatically populate the answers to the medical inquiries.

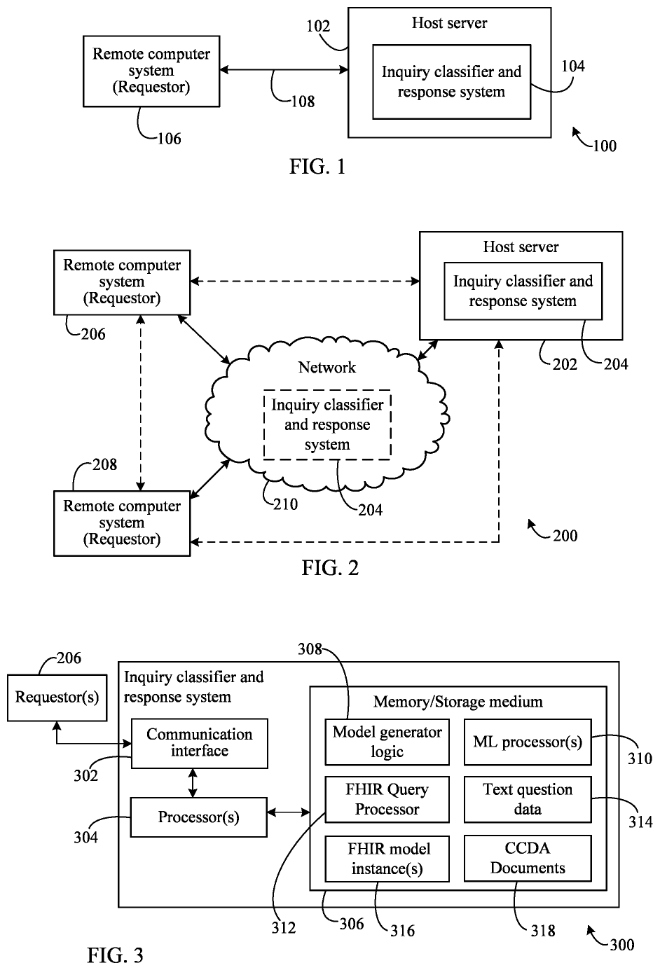

For instance, FIG. 1 shows a block diagram of a network of computer systems 100 that includes an inquiry classifier and response system 104 for classifying and answering medical inquiries based on machine-generated data resources and machine learning models, according to an embodiment. Network of computer systems 100 includes a host server 102 that may include one or more processing devices such as, but not limited to, servers, inquiry classifier and response system 104, and a requestor system 106 that may also be a remote computing system and include one or more processing devices such as, but not limited to, servers and client devices such as laptop/desktop computers and computer terminals, personal handheld devices, etc. Host server 102 may be communicatively coupled or linked to requestor system 106 via a communication link 108.

Requestor system 106 may comprise one or more computers/servers of an entity, such as a trading partner(s), a vendor service(s), a doctor or doctor's office (including nurses and/or other staff), SPOs, PBMs, pharmacies, etc., as noted herein, that desires to request answers to medical inquiries for patients from host server 102 over communication link 108.

Host server 102 may comprise one or more computers/servers of a host entity facilitating access to inquiry classifier and response system 104 by remote computer systems such as requestor system 106, according to embodiments. Host server 102 may include geographically distributed computers/servers, a rack server system(s), a stand-alone server, a cloud-based system, etc. In embodiments, requests of various types from requestor system 106 to host server 102 may cause one or more processes, applications, and/or services to be executed by host server 102, such as but not limited to, ePA requests, etc. In such embodiments, these one or more processes, applications, and/or services may cause host sever 102 to invoke inquiry classifier and response system 104 with the output or result of inquiry classifier and response system 104 being provided back to the invoking process, application, and/or service to be incorporated into a return transmission therefrom.

Communication link 108 may comprise at least one network or direct communication connection, or any combination thereof, between host server 102 and requestor system 106 that enables communication messages such as requests for answers to medical inquiries and CCDA documents, and/or the like, as well as associated responses to such inquiries, as described herein, to be exchanged. As used herein, the term “messages” includes resources such as documents, clinical resources, data, information, packets, and/or the like, related to messaging such as clinical messaging, transmitted and/or received according to any communication standard or protocol, or according to ad hoc communications. In embodiments, communication link 108 may comprise wired and/or wireless portions of one or more networks such as networks of the host entity and requestors, including enterprise networks, the Internet, etc.

Inquiry classifier and response system 104 may comprise hardware and/or software components configured to automatically generate clinical resources such as FHIR models, as described herein, and answers to medical inquiries based on ML models. For instance, in embodiments, inquiry classifier and response system 104 is configured to automatically generate clinical resources, e.g., based on the FHIR standard (FHIR resources), at host server 102 based on a medical inquiry request from requestor system 106, although it is contemplated herein that inquiry classifier and response system 104 is also configured to generate resources without explicit requests therefor. Inquiry classifier and response system 104 is configured to perform this function by utilizing an ontology model (e.g., through RDF) of the FHIR standard with information in one or more documents, e.g., CCDA documents, received from requestor system 106, and/or stored at host server 102. The ontology model is a uniform RDF model for the FHIR standard, in embodiments and as described herein, and provides a characterization of relationships within the FHIR standard, FHIR resources, and FHIR resource values. After a CCDA document is utilized to automatically generate an object model document having information-value pairs for data thereof by inquiry classifier and response system 104, inquiry classifier and response system 104 automatically assigns path definitions for the information-value pairs to map the pairs to objects in the ontology model. Inquiry classifier and response system 104 is configured to then automatically generate an instance model for the FHIR standard that is specific to the CCDA document based on the mapping. The instance model includes corresponding clinical resources according to the FHIR standard for each of the information-value pairs.

Generated instance models may be stored by inquiry classifier and response system 104 in one or more processing devices and/or storage devices described herein. When stored, the instance models may be utilized to answer medical inquiries regarding patients from the requestors or processing devices of the host entity via querying. Additionally, the instance models may be utilized to generate FHIR resources and/or FHIR resource bundles, for subsequent use.

Inquiry classifier and response system 104 is also configured to receive medical inquiries for a patient, as text question data, along with received CCDA documents, from a requestor, e.g., from requestor system 106. Inquiry classifier and response system 104 may include a trained answerability ML model that is trained on prior medical inquiries with parameters directed to question answerability, and another trained ML model that is trained on prior medical inquiries directed to question categories which is utilized to determine categories for received medical inquiries, e.g., via the text question data, from the requestors. Classification for questions as answerable, or not, may be performed in embodiments using a BERT (Bidirectional Encoder Representations from Transformers) model, and/or the like in other embodiments, modified via training. Categories under which medical inquiries that can be answered, as determined by the answerability ML model, may then be determined by another ML mode of inquiry classifier and response system 104 through an analysis of one or more clinical data sources (e.g., CCDA (Consolidated Clinical Document Architecture), FHIR (Fast Healthcare Interoperability Resources) queries, medication histories, fill data, and/or the like) of the patient utilizing the trained ML model for category determination. An ML category model is configured to determine answers for medical inquiries from the appropriate clinical data source based on the determined category(ies). In embodiments, the ML models herein may determine probabilistic or statistical scores/indices in determining answerability, categories, and/or answers for medical inquiries, and these scores/indices may be subsequently used to identify areas for retraining and/or updating the ML models. In embodiments, scores/indices may be required to meet or exceed a threshold value for a positive determinations, and it is contemplated herein that scores/indices within a pre-determined amount of meeting/exceeding such a threshold may be identified for further refinement by the ML models herein. The data and information determined to answer medical inquiries is provided to the requestor at requestor system 106 over communication link 108.

It is also contemplated herein, according to embodiments, that host server 102 and inquiry classifier and response system 104 may together comprise one or more servers that perform the described functionality of inquiry classifier and response system 104, as well as other functionality for a host entity, or may be a single server performing either or both of these types of functionality. Other relational configurations of host server 102 and inquiry classifier and response system 104 are also contemplated herein, as would be understood by a person of skill in the relevant art(s) having the benefit of this disclosure.

Turning now to FIG. 2, a block diagram of a network of computer systems 200 that includes an inquiry classifier and response system 204 is shown, according to an embodiment. Network of computer systems 200 may be a further embodiment of network of computer systems 100 of FIG. 1. Network of computer systems 200 includes a host server 202, inquiry classifier and response system 204, a requestor/remote computer system 206, and a requestor/remote computer system 208. Host provider 202, requestor system 206, and requestor system 208 may be communicatively coupled or linked each other via a network 210.

Host server 202 may be a further embodiment of host server 102 of FIG. 1, and, for the purposes of illustration for FIG. 2, is configured the same, or substantially the same, as host server 102 above. Inquiry classifier and response system 204 may be a further embodiment of inquiry classifier and response system 104 of FIG. 1, and, for the purposes of illustration for FIG. 2, is configured the same, or substantially the same, as inquiry classifier and response system 104 above. Requestor system 206 and requestor system 208 may each be a further embodiment of requestor system 106 of FIG. 1, and for the purposes of illustration for FIG. 2 is configured the same, or substantially the same, as requestor system 106 described above.

Network 210 may be a further embodiment of communication link 108 of FIG. 1. Network 210 may comprise at least one network and/or direct connection (i.e., a communication link), or any combination thereof. That is, network 210 may be any combination of the Internet, a “cloud,” direct communication links, business and/or enterprise networks, and/or the like.

As noted, network 210 is configured to communicatively couple host server 202, requestor system 206, and requestor system 208 to each other. Accordingly, network of computer systems 200 is configured as a further embodiment of network of computer systems 100 in that inquiry classifier and response system 204 is configured to automatically generate FHIR resources and FHIR resource bundles based on CCDA documents received from any system communicatively coupled to inquiry classifier and response system 204, including host server 202, requestor system 206, and requestor system 208, similarly as described above for inquiry classifier and response system 104 of FIG. 1, and to classify and answer medical inquiries based on such machine-generated data resources and on machine learning models.

For instance, an example scenario is now described in the context of network of computer devices 200 as shown in FIG. 2. Host server 202 may receive one or more CCDA documents and/or medical inquiries for a patient from requestor system 206 and/or requestor system 208. Requestor system 206 and/or requestor system 208 may provide one or more CCDA documents and medical inquiries from which inquiry classifier and response system 204 generates FHIR models/resources from which data associated with the patient is determined that is responsive to the inquiries. For example, a health care provider may provide a CCDA document and text question data requesting patient information such as age, a measured body weight, medication names and/or dosages, and/or the like. Subsequently, inquiry classifier and response system 204 may utilize the CCDA document to generate a FHIR instance model corresponding thereto, determine categories for the medical inquiries via a first ML model, map via second ML model the medical inquiries to objects in the FHIR instance model, and query the FHIR instance model to obtain values of the queried-for objects given the mapping. The information in the query result may then be provided back to the appropriate requestor system. As noted above, a third ML model (e.g., trained on a set of prior inquiries for answerability) may be implemented to determine if a given medical inquiry is answerable by the system prior to the first ML model determining the categories. It should be noted herein that the description of ML models as “first,” “second,” “third,” etc., is not limiting with respect to ordering of implementation during operations of systems for embodiments, efficacy of answerability determinations, category determination, answers to inquiries, and/or the like, but rather is utilized in this description to differentiate ML models from each other in embodiments.

Still referring to FIG. 2, while shown for illustrative simplicity and brevity as including a single host provider (e.g., host server 202) and two remote computer systems (requestor systems 206/208), it is contemplated herein that network of computer systems 200 may include more or fewer of any of these components in embodiments.

The described techniques and embodiments provide for automatically generating clinical observation resources, e.g., ontology models in accordance with the FHIR format, from documents that are not in the FHIR format, e.g., CCDA documents. Systems, devices, and apparatuses contemplated herein, such as systems, devices, and apparatuses that include inquiry classifier and response systems and/or components thereof, may be configured in various ways for generating clinical resource information objects according to the described techniques and embodiments, e.g., from CCDA documents, and querying specific ones of those objects to answer medical inquiries in text data by classification and mapping via ML models. The described embodiments and techniques also allow for centralization of medical inquiry handling based on ML models, with the additional improvements provided from the ML models being trained/retrained using information/inquiries from a variety of unrelated sources which allows for a rich model training/retraining environment in additional to system and network resource efficiencies through such centralization. The described embodiments and techniques may be configured and scaled to accommodate multiple CCDA documents, as well as different document formatting standards, such as, but not limited to, CCDA and FHIR.

As noted above, embodiments for systems and devices may be configured to perform their functions and operations (e.g., methods) in various ways, and it is contemplated herein that in various embodiments, one or more components described and/or shown may not be included and that additional components may be included.

Turning now to FIG. 3, a block diagram of an inquiry classifier and response system 300 is shown, according to an embodiment. Inquiry classifier and response system 300 may be a further embodiment of inquiry classifier and response system 104 of FIG. 1 and/or inquiry classifier and response system 204 of FIG. 2. That is, inquiry classifier and response system 300 may be included or implemented in a host server, e.g., host server 102 of FIG. 1 and/or host server 202 of FIG. 2, that is communicatively coupled to one or more remote computer systems over a communication link or network, as described above.

Inquiry classifier and response system 300 includes a communication interface 302, one or more processors (“processor”) 304, and a memory/storage medium (“memory”) 306. Processor 304 is communicatively coupled to communication interface 302 and to memory 306. Communication interface 302 is configured to be communicatively coupled to a communication link and/or to a network, such as communication link 108 of FIG. 1 and/or network 210 of FIG. 2 for communication with one or more remote devices such as requestors as described herein. As exemplarily illustrated, requestor system 206 as described for FIG. 2 is communicatively coupled.

Communication interface 302 may be one or more interfaces, such as hardware network interfaces, that are configured to transmit and/or receive communications and messages such as requests, as well as documents, text data, and/or the like, over a network or communication link as described herein.

Processor 304 and memory 306 may respectively be any type of processor circuit(s)/system(s) and memory that is described herein, and/or as would be understood by a person of skill in the relevant art(s) having the benefit of this disclosure. Processor 304 and memory 306 may each respectively comprise one or more processors or memories, different types of processors or memories (e.g., at least one cache for query processing), remote processors or memories, and/or distributed processors or memories. Processor 304 may be multi-core processors configured to execute more than one processing thread concurrently. Processor 304 may comprise circuitry that is configured to execute and/or process computer program instructions such as, but not limited to, computer program instructions to perform the described embodiments, including functions, operations, and methods for classifying and answering medical inquiries based on machine-generated data resources and machine learning models, including one or more of the components thereof as described herein, which may be implemented as computer program instructions, as described herein. For example, in performance of/operation for any flowcharts, flow diagrams, processes, operations, etc., herein, processor 304 may execute program instructions as described.

Memory 306 includes volatile storage portions such as a random access memory (RAM) and/or persistent storage portions such as hard drives, non-volatile RAM, and/or the like, to store or be configured to store computer program instructions/code for classifying and answering medical inquiries based on machine-generated data resources and machine learning models, as described herein, as well as to store other information and data described in this disclosure including, without limitation, components, data/information, and/or the like shown in FIG. 3, as well as those in shown for different embodiments. For example, as shown in FIG. 3, memory 306 is configured to store model generator logic 308, a ML processor 310, and an FHIR query processor 312. Memory 306 is also configured to store a text question data 314 (e.g., having medical inquiries) that may be received from a requestor system, one or more of FHR model instances 316, and one or more of CCDA documents 318. In embodiments, one or more of text question data 314, FHR model instances 316, and/or CCDA documents 318 may not be stored at all times in memory 306, but may be stored upon generation or receipt thereof, as described herein. It is also contemplated herein that, in embodiments, FHR model instances 316 may respectively be models based on a framework or data structure implementation other than RDF. Memory 306 is also configured to store standard RDF models, stand FHIR models, mapping logic/extensions, object model documents corresponding to CCDA documents with CCDA documents 318, and/or the like, as described herein.

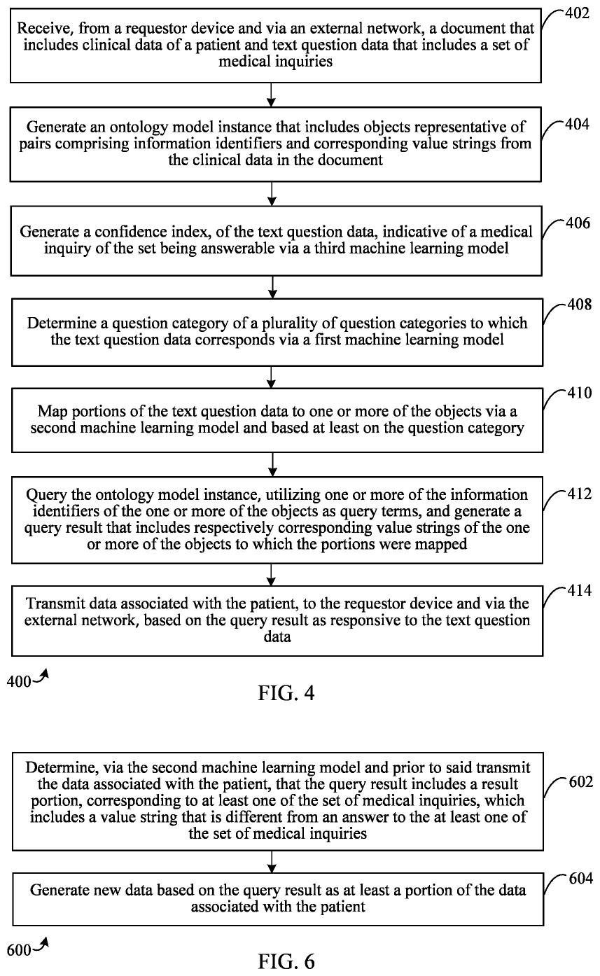

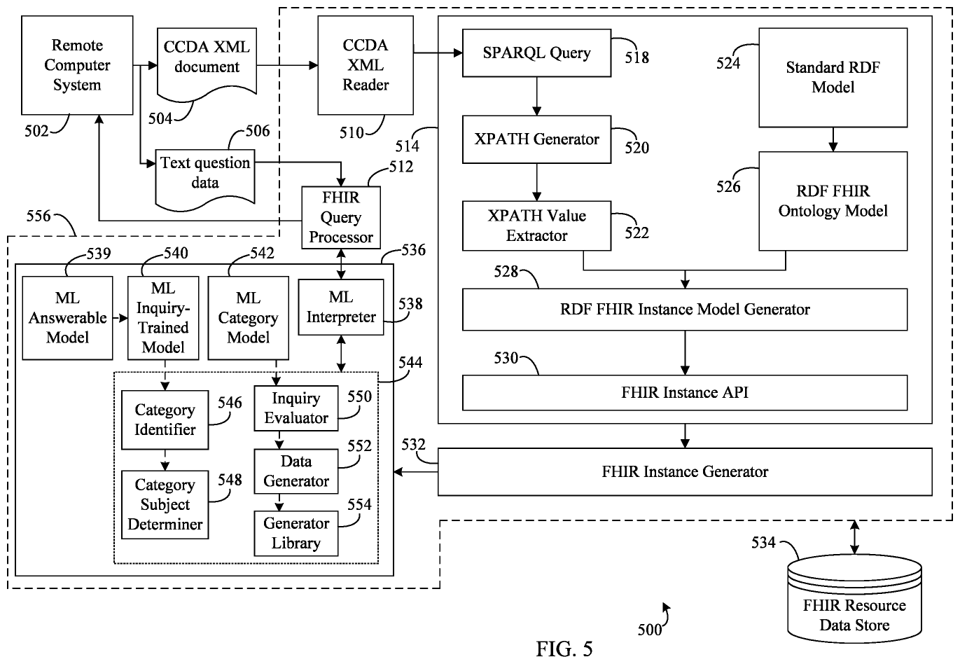

The components of inquiry classifier and response system 300 are discussed in further detail below with respect to FIG. 4 and FIG. 5.

In FIG. 4, a flowchart 400 for classifying and answering medical inquiries based on machine-generated data resources and machine learning models is shown, according to an embodiment. That is, flowchart 400 may exemplify a method performed in or by a computing system for classifying and answering medical inquiries based on machine-generated data resources and machine learning models. Example techniques and embodiments described herein may be configured and/or implemented to perform various aspects classifying and answering medical inquiries based on machine-generated data resources and machine learning models according to flowchart 400. For instance, inquiry classifier and response system 104 of FIG. 1, inquiry classifier and response system 204 of FIG. 2, and/or inquiry classifier and response system 300 of FIG. 3, along with any of their respective subcomponents, or associated system-wide components, may perform functions according to flowchart 400 of FIG. 4.

In FIG. 5, a system 500 execution flow for classifying and answering medical inquiries based on machine-generated data resources and machine learning models is shown, according to an embodiment. System 500 is described with respect to FIGS. 3 and 4. In embodiments, system 500 may include an inquiry classifier and response system 556 which may be a further embodiment of inquiry classifier and response system 300 of FIG. 3.

Flowchart 400 of FIG. 4 and system 500 of FIG. 5 are described as follows in the context of inquiry classifier and response system 300 of FIG. 3 for exemplary illustration and with respect to each other.

Flowchart 400 begins at step 402. In step 402, a document that includes clinical data of a patient and text question data that includes a set of medical inquiries is received from a requestor device and via an external network. For example, with respect to FIG. 3, a remote computer system (e.g., a requestor such as requestor system 206) may provide a document having clinical data and text associated with medical inquiries which may be received over a network by communication interface 302 of inquiry classifier and response system 300. That is, the document may be structured/formatted according to CCDA standards in embodiments and includes clinical data associated with a patient. The text question data comprises text that represents medical inquiries associated with the patient, as exemplarily described herein. The text question data may be provided serially, concurrently, or in any other relational manner with respect to the document. In embodiments, the text question data may comprise a file, document, character payload of a message packet, etc. Communication interface 302 is configured to provide received document and text question data to processor 304 and/or to memory 306.

Referring also now to FIG. 5, as illustrated in system 500, a CCDA XML document (“document”) 504 is provided to a CCDA XML reader (“reader”) 510 from a remote computer system 502, which may be a further embodiment of a requestor system described above in FIGS. 1-3, to inquiry classifier and response system 556. Additionally, text question data 506 is provided by remote computer system 502 to inquiry classifier and response system 556. For instance, communication interface 302 shown in FIG. 3, not shown in FIG. 5 for illustrative clarity, is configured to receive document 504 and text question data 506, and to provide document 504 and text question data 506 for processing in system 500. As noted, processor 304, described above with respect to FIG. 3, is configured to execute computer program instructions (i.e., computer program logic) stored in memory 306. A FHIR execution block 514 is shown in system 500 as representing the execution of computer program instructions for ontology model generation, and is an embodiment of model generator logic 308 in FIG. 3. FHIR execution block 514 may be executed by processor 304.

Referring back to flowchart 400 of FIG. 4, in step 404, an ontology model instance is generated that includes objects representative of pairs comprising information identifiers and corresponding value strings from the clinical data in the document. For instance, referring also to FIGS. 3 and 5 for illustration, when document 504 is received by communication interface 302 and loaded into memory 306, processor 304 may be configured to activate reader 510 from object model document logic when loaded into memory 306 to cause reader 510 to generate an object model document based on document 504. As an example, consider a scenario where an information-value pair for a clinical observation of body weight for the patient is provided in text question data 506 as a medical inquiry. Document 504 may include an entry therefore, e.g., identifier: BODY WEIGHT (MEASURED); value string: “144.84”, and reader 510 is configured in this example to generate the object model document to include this information-value pair, as well as information-value pairs for the given name and the family name of the patient: e.g., given, “Paulina”; family: “Coffin”; and/or the like for any other clinical information in document 504.

Continuing with the generation of the ontology model instance, path definitions are assigned for each of the information-value pairs that define mappings between the information-value pairs in the object model document and objects of an ontology model (e.g., an FHIR ontology model). For instance, processor 304 is configured to load into memory 306, and activate, mapping logic to cause the assignment of path definitions for each information-value pair. A base RDF model, e.g., a standard RDF model, is loaded into memory 306 as shown in a standard RDF model 524 in FHIR execution block 514, and is augmented with defined mappings and/or a model of extension classes, e.g., from stored mapping logic/extensions, also loaded into memory 306, for defining the available mappings between the information-value pairs in the object model document and objects of standard RDF model in a second standard format (in embodiments, the standard RDF model may be a FHIR model). The augmented model having the class extensions and/or defined mappings is shown in FHIR execution block 514 as an RDF FHIR ontology model 526. In some embodiments, RDF models and extensions as described herein may be implemented according to the “OWL” Web Ontology Language (see https://www.w3.org/TR/owl-ref/), and/or equivalents.

SPARQL, a recursive acronym identifying the SPARQL Protocol and RDF Query Language, may be utilized in embodiments for XPATH generation. The mapping logic assigns the path definitions such as XPATH definitions between each of the information-value pairs of the object model document and the appropriate, available object of the augmented RDF model by first querying RDF FHIR ontology model 526 using SPARQL query logic 518 which may be included with the mapping logic/extensions, although other query protocols are contemplated herein. SPARQL query logic 518 is configured to provide queries against models, according to embodiments, such as RDF ontology models as described herein. That is, in embodiments, SPARQL query logic 518 is configured to query the augmented RDF model, RDF FHIR ontology model 526, for defined mappings of the standard model components and properties (including, e.g., “child” properties to which the actual values, i.e., codings, are mapped in the FHIR model (“CodeableConcept.coding”), which may then have objects generated therefrom, e.g., Java™ objects, that are provided to XPATH generator logic 520.

XPATH generator logic 520 may also be included in the mapping logic/extensions. XPATH generator logic 520 is configured to generate the paths between each model component of RDF FHIR ontology model 526 and the information-value pairs of the object model document by linking the mappings of the model components and properties with CCDA codes (such as but not limited to Logical Observation Identifiers Names and Codes (“LOINC”®)) of the information-value pairs. In the prior example, the BODY WEIGHT (MEASURED) identifier is associated with LOINC code “3141-9” which is in turn associated with a LOINC code of 8716-3 for “Vital signs,” and so on. XPATH generator logic 520 links the LOINC code “3141-9” for BODY WEIGHT (MEASURED) with the appropriate defined mapping for clinical observations in RDF FHIR ontology model 526.

Continuing with this example, the value property (“Val”) may be given as “/entry/organizer/component/observation/code.” This is interpreted by the executing logic such that for observation entries in a CCDA Vital Signs section (when identified in the CCDA by LOINC “8716-3”), the code for each included observation can be found by following the XPATH “/entry/organizer/component/observation/code” (i.e., the value in Val). Furthermore, the CCDA entry specifies that the actual code value relates to the Observation.code property of the FHIR Observation resource. These are the “parent” class and the “parent” property in which the code should be placed within the FHIR model, as described herein. It should also be noted that FHIR model components and properties for clinical observations are not a simple strings (i.e., the code value). Rather, the Observation.code is another FHIR datatype object called CodeableConcept. A CodeableConcept consists of a Coding (another FHIR datatype) and a text (string) property. Ultimately, the code for an observation from the CCDA becomes the value of the Coding.code property of an object that is referenced by a CodeableConcept.coding property in a FHIR model instance, which is referenced by an Observation.code property. As an illustrative, non-limiting example, a FHIR Coding object “A” is created whereby A. code=3141-9, and A. display=BODY WEIGHT (MEASURED). Moreover, a FHIR CodeableConcept object “B” is created, whereby B. coding=A (i.e., the Coding object). Finally, a FHIR Observation object “C” is created, which among other things has its coding property set to B (i.e., C.coding=B). Example XPATH definitions according to the above examples may be:

```
. . .
, [P:Observation] [PProp:Observation.code]
[PVal:/entry/organizer/component/observation/code]
[CProp:CodeableConcept.coding]
  [Val:/entry/organizer/component/observation/code]
  [Assoc: [10153-2, 10155-0, 10157-2, 11384-5, 11450-4, 11496-7,
  11502-2, 29762-2, 30954-2, 48765-2, 8716-3]]
. . .
, [P:Composition] [PProp:Composition.subject]
  [PVal:/recordTarget/patientRole] [CProp:Patient.name]
  [Val:/patient/name]
, [P:Patient] [PProp:Patient.name] [PVal:/patient/name]
  [CProp:HumanName.family] [Val:/family]
, [P:Patient] [PProp:Patient.name] [PVal:/patient/name]
  [CProp:HumanName.given] [Val:/given]
, [P:HumanName] [PProp:HumanName.family] [PVal:/family]
  [Val:/family]
, [P:HumanName] [PProp:HumanName.given] [PVal:/given] [Val:/given
. . .
```

Next, XPATH value extractor logic 522, which may be included in the mapping logic/extensions, is configured to utilize the XPATH definitions to create the explicit maps that relate a value of an information-value pair in a CCDA document to the property in the FHIR model to which the value is associated. Because various sections of a CCDA document, all containing observations, can be organized by different structures, it is necessary to assemble all XPATHs in which an Observation may occur. For example, an Observation in one section of a CCDA may be contained within organizer/composition components, but in another section be contained in act/entryRelationship components, and in yet other sections, neither. Hence, the embodiments described herein provide for coverage of all the structures in a CCDA document within which an Observation may occur. Accordingly, XPATH value extractor logic 522 is configured to create the explicit maps by iteratively assembling all possible XPATH definitions.

XPATH value extractor logic 522 is configured to utilize the explicit maps to take the CCDA code for weight, and store it in a FHIR Coding object (Coding.code) property using ‘breadcrumb’ information, as shown below. Further, if read in reverse order, the constructed map provides that this Coding object is referenced by a CodeableConcept (via the CodeableConcept.coding), which is in turn referenced by an Observation (via Observation.code), which is one of (i.e., referenced by) the entries in a particular section (via SectionComponent.entry). Therefore, the CCDA construction of:
ns:section/ns:entryins:organizer/ns:component/ns:observation/ns:co de/@code [CCDA code value],
is mapped to FHIR as:
SectionComponent.entry=>Observation.code=>CodeableConcept.coding=>Coding.code,
according to:

```
[P:SectionComponent] [PProp:SectionComponent.entry] [PVal:section]
  [CProp:Coding.code]
  [Val:ns:section/ns:entry/ns:organizer/ns:component/ns:observati
  on/ns:code/@code] [Res:observation] [Assoc:[10153-2, 10155-0,
  10157-2, 11384-5, 11450-4, 11496-7, 11502-2, 29762-2, 30954-2,
  48765-2, 8716-3]] [Bread:[SectionComponent=
  SectionComponent.entry, Observation=Observation.code,
  CodeableConcept=CodeableConcept.coding, Coding=Coding.code]].
```

Accordingly, XPATH value extractor logic 522 is configured to extract the values of the information-value pairs of the CCDA document, document 504.

An instance model, of the RDF ontology model, that is representative of the document and that is in the second standard format based on the path definitions is then generated. For example, RDF FHIR instance model generator logic (“instance model generator logic”) 528 which may be a part of model generator logic 308 of FIG. 3, is configured to generate an RDF FHIR instance model representative of the CCDA document, document 504 (although other formats in addition to FHIR are contemplated herein). In embodiments, the FHIR instance model may be stored in memory 306 of FIG. 3 as FHIR model instance(s) 316. Instance model generator logic 528 is configured to dynamically assemble the RDF FHIR instance model based on RDF FHIR ontology model 526, and the XPATH mapping and the extracted values noted above. According to some embodiments, for one or more, or each, value extracted from document 504, as described above, instance model generator logic 528 is configured to assemble a number of corresponding FHIR objects that will be associated with the CCDA values. That is, instance model generator logic 528 assembles the RDF FHIR instance model to generate a specific FHIR instance that corresponds to the values for patient and clinical information, such as but not limited to observations, included in document 504.

Additionally, objects for the instance model are created, and values are provided to the objects for the instance model corresponding to each of the at least one information-value pair by instance model generator logic 528. Instance model generator logic 528 is configured to create instance model objects, e.g., for the FHIR instance model, based on standard RDF FHIR model and the XPATH mapping described above. The standard RDF FHIR model may include a standard resource kind having objects, e.g., for each of Observation and Patient, in addition to others, as would be apparent to those of skill in the relevant art(s) having the benefit of this disclosure. In the example herein, an observation of body weight measured for a patient is described. Accordingly, a FHIR object for the patient and a FHIR object for the observation would be assembled from standard RDF FHIR model for the for the FHIR instance model based on the XPATH mapping, while other standard FHIR objects may be excluded from the assembly of the FHIR instance model.

In examples where two observations are included for a patient in a CCDA document, two instances of the FHIR observation object of the RDF FHIR model would be used to assemble the FHIR instance model, likewise for three or more observations, as well as for other types of FHIR resources and their objects. In this manner, the RDF FHIR model serves as a standard template that is utilized for any number of a given FHIR resource that is required for generating objects in a FHIR instance model.

It should also be noted that in embodiments, the specific FHIR instance may include only resources and objects that correspond to the information present in document 504 for which the specific FHIR instance is representative, and/or may include a subset of resources and objects that is less than all of the resources and objects in the standard FHIR model.

In providing values or codings to the objects for the FHIR instance model that correspond to each information-value pair(s) of the CCDA document, e.g., document 504, the values of the information-value pairs extracted by XPATH value extractor logic 522 based on the XPATH mappings, as described above, are assigned to the corresponding objects in the FHIR instance model by instance model generator logic 528. Values/codings may be assigned as objects are assembled for the FHIR instance model or subsequent to the assembly.

Resources are then generated in accordance with the second standard format from the objects for the instance model. For example, the FHIR instance model generated based on the CCDA document (e.g., document 504 having a first format, CCDA) in (404) by instance model generator logic 528 has a FHIR standard format, e.g., a second format that is different from CCDA, although other standard formats are contemplated herein. As noted above, generated FHIR instance models may be stored in memory 306 of FIG. 3 as FHIR model instance(s) 316. A FHIR instance model generated, or generated and stored, is provided by a FHIR instance application programming interface (“API”) 530 to FHIR instance generator logic 532 which is configured to extract one or more instance values/definitions from FHIR instance models to generate individual FHIR resource objects, also FHIR ontology model instances herein which are specific to document 504 and the patient associated therewith.

These generated FHIR resource objects or FHIR ontology model instances may be stored in a FHIR resource data store 534. FHIR resource data store 534 may comprise, e.g., one or more databases, and may be a central or distributed data store. FHIR resource data store 534 may be hosted by a service provider of a system as described herein, or may be hosted by a third-party provider that grants access to FHIR resource data store 534 for such systems. FHIR resource data store 534 may be a part of a system such as inquiry classifier and response system 300 of FIG. 3 or inquiry classifier and response system 556 of FIG. 5, or may be a separate component(s) that are accessible by such systems described herein over a network or other communication connection.

Additionally, generated FHIR resource objects may be stored in FHIR resource data store 534 as groups based on a patient. For example, when more than one FHIR resource objects are generated, as described herein, and stored in FHIR resource data store 534, the FHIR resource objects may be grouped, tagged, indexed, etc., according to the patient associated therewith. In such embodiments, access efficiency for these FHIR resource objects may be increased. As another example, newly-generated FHIR resource objects may be added to groups of existing, previously-generated FHIR resource objects stored in FHIR resource data store 534 based on commonality, such as but without limitation, a given patient. In embodiments, a FHIR instance model and/or FHIR resource objects may be generated responsive to a request or query therefor, and/or responsive additional information being required by an application, process, service, etc., of a host server comprising inquiry classifier and response system 556. It is also contemplated herein that some such requests or queries may specify a number of observations, values, or other information for a FHIR instance model that is less than the total number of observations, values, or other information contained in the CCDA document. FHIR instance models generated, or generated and stored, that are provided by API 530 to FHIR instance generator logic 532 as noted above, may be provided based on a request or a query.

Flowchart 400 of FIG. 4 continues in step 406, in which a confidence index, of the text question data, is generated that is indicative of a medical inquiry of the set being answerable via an initial machine learning model. For instance, text question data 314 in FIG. 3 may include one or more medical inquiries, as described herein, and portions of text question data 314, e.g., each comprising a medical inquiry, are determined as being answerable or not via a respective confidence index determined by a ML model, in embodiments, as described herein.

Referring again to FIG. 5, text question data 506 may be provided to a FHIR query processor 512 (e.g., an embodiment of FHIR query processor 312 in FIG. 3) that is configured to query FHIR model instances, e.g., FHIR model instances and FHIR ontology model instances for results comprising specific clinical information stored in the FHIR standard format that corresponds to answers for the medical inquiries in text question data 506. FHIR query processor 512 may be a separate hardware-based processor, or may be a program or application executing on a processor(s) of systems described herein. FHIR query processor 512 utilizes an ML processor block 536 which may be an embodiment of ML processors 310 in FIG. 3. ML processor block 536 includes an ML interpreter 538 configured to pre-process and/or clean raw text question data 506 prior to determining categories for medical inquiries thereof and performing queries against the FHIR ontology model instances. In embodiments, FHIR query processor 512 may perform these operations. The pre-processing and/or cleaning may be performed by a natural language processor based on text question data 506 including natural language for the medical inquiries. ML processor block 536 includes an ML answerable model 539 configured to generate the confidence index for a medical inquiry in a portion of text question data 506 where the confidence index indicates whether or not the medical inquiry is answerable by the system. In embodiments, ML answerable model 539 may be a BERT model that is trained as described herein. In embodiments, if a medical inquiry in a portion of the text question data is not answerable, an alternative data source may be sought, or that particular medical inquiry may be skipped. When a medical inquiry is determined to be answerable, it is passed on to another ML model for category determination as described below.

Flowchart 400 of FIG. 4 continues in step 408, in which a question category of a plurality of question categories to which the text question data corresponds is determined via a first machine learning model. For example, text question data 314 in FIG. 3 may include one or more medical inquiries, as described herein, and portions of text question data 314, e.g., each comprising a medical inquiry, are determined as corresponding to a question category, in embodiments.

Referring again to FIG. 5, ML processor block 536 also includes an ML inquiry-trained model 540 that is utilized by ML interpreter 538 to determine the corresponding question category for the medical inquiry portions of text question data 506. As illustrated with respect to both FIGS. 4 and 5, when ML answerable model 539 determines, based on the confidence index, that a medical inquiry is answerable, ML inquiry-trained model 540 next determines the category for the inquiry. In embodiments, medical inquiries having corresponding, identified question categories are answerable via a query of the FHIR ontology model instances by FHIR query processor 512 (medical inquiries that do not have a corresponding question category may be answered by other operations described in further detail below).

An ML execution block 544 is shown in system 500 as representing the execution of computer program instructions for utilization of ML models and processing according to embodiments herein. ML execution block 544 may be executed by processor 304 running ML interpreter 538. For instance, a category identifier 546 and a category subject determiner 548 are included in ML execution block 544 and may utilize ML inquiry-trained model 540. Category identifier 546 identifies a corresponding question category for a medical inquiry in text question data 506, and may identify the question category based on statistical or probabilistic scores using ML inquiry-based model 540. Category subject determiner 548 determines the subject of the identified question category for mapping the medical inquiry to an object(s) of the FHIR ontology model instance. In embodiments, categories to be determined, and their subjects, may be associated with different types/classes of objects in a standard FHIR model or base RDF model that comprises supported objects for inclusion in any FHIR ontology model instance.

Flowchart 400 of FIG. 4 continues in step 410, in which portions of the text question data are mapped to one or more of the objects via a second machine learning model and based at least on the question category. For example, as noted herein, text question data 314 in FIG. 3 may include one or more medical inquiries where portions of text question data 314 each comprise a medical inquiry, and these medical inquiries are mapped to corresponding objects of the FHIR ontology model instance based on the identified question category and/or question category subject, according to embodiments. That is, subsequent to the question category and subject being determined and identified, an ML category model 542 of ML block 536 in FIG. 5 is configured to perform such a mapping based on the question category identified. In other words, identified question categories are mapped using ML category model 542, thus binding the identified question categories to the corresponding FHIR model generation concepts enabling inquiry classifier and response system 556 of system 500 to make the connections between the medical inquiries and use the FHIR resources to query for the relevant data that is provided in the patient's CCDA document.

Flowchart 400 of FIG. 4 continues in step 412, in which the ontology model instance is queried utilizing one or more of the information identifiers of the one or more of the objects as query terms, and a query result that includes respectively corresponding value strings of the one or more of the objects to which the portions were mapped is generated. For example, and also with respect to FIG. 5, FHIR query processor 512 is configured to query an FHIR ontology model instance, as generated for document 504 and described above, provided to ML processor block 536 based on the mappings generated in step 410 of flowchart 400 and the information identifiers of the objects in the FHIR ontology model instance via the performance of FHIR query functions. The query result is returned with the value strings associated with the information identifiers of the relevant objects. Value strings in the query result that answer the medical inquiries of text question data 506 are thus ready to be returned to the requestor.

Flowchart 400 of FIG. 4 continues in step 414, in which data associated with the patient is transmitted to the requestor device and via the external network based on the query result as responsive to the text question data. For example, upon receiving a query result at FHIR query processor 512 for medical inquiries of text question data 506, the data of the query result, i.e., data associated with the patient, is provided to the requestor, e.g., remote computer system 502, an application, process, and/or service executing at the host server comprising inquiry classifier and response system 556, etc.

In some embodiments, such as when a query of a FHIR ontology model instance does not return results for query by FHIR query processor 512 that answer a medical inquiry of text question data 506, by example and not limitation, when an age is requested in a medical inquiry but a date of birth is returned, additional ML processing may be performed via an inquiry evaluator 550, a data generator 552, and a generator library 554, of ML execution block 544.

For instance, turning now to FIG. 6, a flowchart 600 for classifying and answering medical inquiries based on machine-generated data resources and machine learning models is shown, according to an embodiment. Embodiments described herein may be configured and/or implemented to perform various aspects thereof according to flowchart 600, which may be a further embodiment of flowchart 400 in FIG. 4 (e.g., steps subsequent to step 412 and prior to step 414). Flowchart 600 is described as follows in the context of inquiry classifier and response system 500 of FIG. 5 for exemplary illustration.

Flowchart 600 begins at step 602. In step 602, it is determined, via the second machine learning model and prior to said transmit the data associated with the patient, that the query result includes a result portion, corresponding to at least one of the set of medical inquiries, which includes a value string that is different from an answer to the at least one of the set of medical inquiries. For example, also referring to FIG. 5, ML category model 542 is configured to determine if the query result from step 412 in flowchart 400 includes a value string for a specific medical inquiry that does not provide an answer thereto. That is, there are scenarios contemplated herein, such as the age inquiry above that returns a date of birth, in which the query result data is related to, but does not specifically answer, the medical inquiry. In such scenarios, category model 542 is configured to determine that new data is required to fully answer the medical inquiry via inquiry evaluator 550 of ML execution block 544. Inquiry evaluator 500 may determine by context, value string form, value string type, and/or the like that this portion of the query result is not the data sought in the medical inquiry of text question data 506.

Flowchart 600 continues at step 604, in which new data is generated based on the query result as at least a portion of the data associated with the patient. For example, data generator 552 of ML execution block 544 is configured to generate new data that answers the medical inquiry based on generator library 554. That is, utilizing ML category model 542 and the returned value string, data generator 552 determines from generator library 554 how to generate the new data based on the returned value string and the expected value. The new data generated by data generator 552 is provided as part of the patient data, instead of the value string returned in the query result, for provision to the requestor in step 414 of flowchart 400.

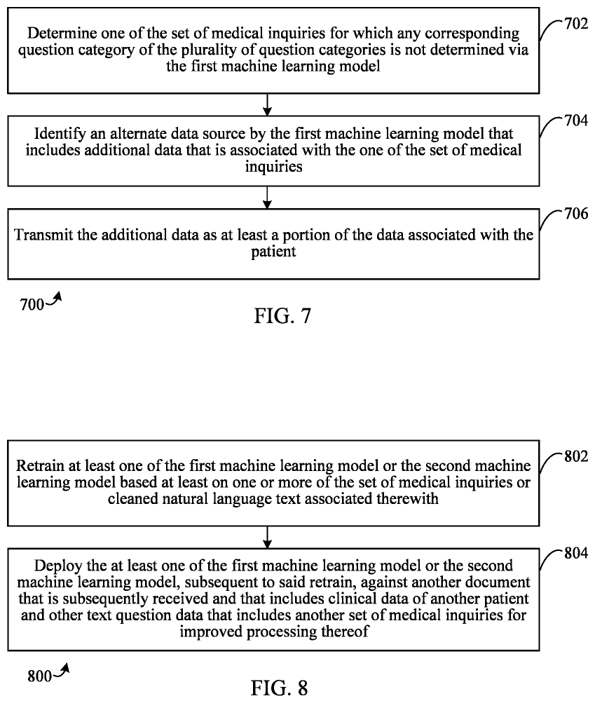

In FIG. 7, a flowchart 700 for classifying and answering medical inquiries based on machine-generated data resources and machine learning models is shown, according to an embodiment. Embodiments described herein may be configured and/or implemented to perform various aspects thereof according to flowchart 700, which may be a further embodiment of flowchart 400 in FIG. 4 (e.g., steps subsequent to step 408 and prior to step 410). Flowchart 700 is described as follows in the context of inquiry classifier and response system 500 of FIG. 5 for exemplary illustration.

Flowchart 700 begins at step 702. In step 702, it is determined that one of the set of medical inquiries for which any corresponding question category of the plurality of question categories is not determined via the first machine learning model. For example, as noted above with respect to FIG. 5, ML inquiry-trained model 540 is utilized to determine corresponding question categories for the medical inquiry portions of text question data 506, which may be based initially on a determination by ML answerable model 539 determining, via a confidence index, that the question is answerable. However, in some embodiments, there may not be a corresponding question category for a medical inquiry included in text question data 506. In such cases, ML inquiry-trained model 540 is configured to make such a determination. As a non-limiting example, a medical inquiry may be provided that corresponds to a new treatment or prescription drug, or to a prescription fill, that is not represented in the CCDA document provided, e.g., document 504, and/or in association with objects of a model as described herein.

Flowchart 700 continues at step 704, in which an alternate data source is identified by the first machine learning model that includes additional data that is associated with the one of the set of medical inquiries. For example, ML inquiry-trained model 540 may be configured to determine an alternate data source for data that would satisfy the medical inquiry for which a question category is not available. Such alternative sources may include other stored CCDA documents, other FHIR instance models, as well as pharmacy fill data for the patient that is previously collected and locally stored by memory 306 in FIG. 3, by way of example, and/or the like.

Flowchart 700 continues at step 706, in which the additional data is transmitted as at least a portion of the data associated with the patient. For example, required data obtained from the alternative data source, e.g., by FHIR query processor 512 in conjunction with the operations of FHIR execution block 514 for another stored CCDA document, processor 304 in FIG. 3 by retrieval from storage, etc., may be provided to the requestor as part of step 414 of flowchart 400 described above for FIG. 4.

In FIG. 8, a flowchart 800 for classifying and answering medical inquiries based on machine-generated data resources and machine learning models is shown, according to an embodiment. Embodiments described herein may be configured and/or implemented to perform various aspects thereof according to flowchart 800, which may be a further embodiment of flowchart 400 in FIG. 4. Flowchart 800 is described as follows.

Flowchart 800 begins at step 802. In step 802, at least one of the first machine learning model or the second machine learning model is retrained based at least on one or more of the set of medical inquiries or cleaned natural language text associated therewith. For example, as described herein, there may be included in text question data one or more medical inquiries that do not correspond to a question category by which an FHIR query processor is enabled to query an FHIR ontology model instance based on a provided CCDA document (e.g., new treatments or prescription drugs, new diagnoses, prescription fills, or any other previously un-modeled information that is collected from the body of requestors). When a medical inquiry does not correspond to a question category, that inquiry may be marked or otherwise noted as a potential parameter for updating and/or retraining ML inquiry-trained model 540 and/or ML category model 542.

Flowchart 800 continues at step 804, in which the at least one of the first machine learning model or the second machine learning model is deployed, subsequent to said retrain, against another document that is subsequently received and that includes clinical data of another patient and other text question data that includes another set of medical inquiries for improved processing thereof. For example, ML inquiry-trained model 540 and/or ML category model 542, after retraining in step 802, may be deployed against future requests in which CCDA documents and text question data are received. In this way, utilizing a central processing location and varied sources of new parameters for ML model updating, the embodiments herein allow for a unique framework by which evolving ML models are utilized and improved.

It is also contemplated herein that an answerability ML model described herein, such as ML answerable model 539 shown in FIG. 5, may be similarly trained/retrained and/or deployed according to flowchart 800 based on one or more medical inquiries, cleaned natural language text associated, and/or the like.

### III. Example Ontology Model Embodiments

As described above, systems may be configured in various ways to perform their described functions. For instance, systems may be configured to generate FHIR resources for clinical information (e.g., that is contained in CCDA documents) for patients where the information is associated with, but not limited to, patient identification, practitioners, appointments, clinical observations, clinical documents, medications, accounts, and/or the like, as defined by the FHIR standard. An extensive list of resources for FHIR is not provided here for the sake of brevity, however, it is contemplated that any FHIR resources may be similarly or analogously treated and/or modeled according to the examples described herein.

Modeling based on metadata that defines the structure and properties of FHIR resources in RDF may be performed according to the described techniques and embodiments. The RDF modeling allows the described embodiments to generate FHIR instance models using desired information from CCDA documents and mappings to standard RDF models, as these models are structured to provide for versatile, targeted queries for clinical resource attainment. For example, a “Patient” resource for FHIR may be modeled based on its metadata to allow the generation of an RDF model for the resource that includes dependent resources such as, but without limitation, vital signs and body weight. The following is an example, non-limiting code segment represented in “.ttl” format, e.g., in a terse RDF triple language (“TTL”), showing a compact text form of a graphical RDF model:

| | |
|---|---|
| @prefix CCDA: | <http://hostProvider.org/CCDA#> . |
| @prefix rdf: | <http://www.w3.org/1999/02/22-rdf-syntax-ns#> . |
| @prefix owl: | <http://www.w3.org/2002/07/owl#> . |

@prefix HostProviderFHIR:
<http://hostProvider.org/HostProviderFHIR#> .

| | |
|---|---|
| @prefix xsd: | <http://www.w3.org/2001/XMLSchema#> . |
| @prefix rdfs: | <http://www.w3.org/2000/01/rdf-schema#> . |

CCDA:Observation_1f20e739-e1db-49b3-ba0b-3ff1982dd765

| | |
|---|---|
| a | <http://hostProvider.org/FHIR#Observation> ; |

<http://hostProvider.org/FHIR#Observation.code>

CCDA:CodeableConcept_1d48f00c-f444-40ae-8a1e-

fde07a054010 .
CCDA:CodeableConcept_1d48f00c-f444-40ae-8a1e-fde07a054010

| | |
|---|---|
| a | <http://hostProvider.org/FHIR#CodeableConcept> ; |

<http://hostProvider.org/FHIR#CodeableConcept.coding>

CCDA:Coding_87fb56c3-cf55-4a42-b49e-4df7e7ad772e .

CCDA:Coding_87fb56c3-cf55-4a42-b49e-4df7e7ad772e

| | |
|---|---|
| a | HostProviderFHIR:Coding ; |

<http://hostProvider.org/FHIR#Coding.code>

“3141-9” ;

<http://hostProvider.org/FHIR#Coding.display>

“BODY WEIGHT (MEASURED)” .

CCDA:Patient_758af88b-d467-45f4-9907-56896e5480f4

| | |
|---|---|
| a | <http://hostProvider.org/FHIR#Patient> ; |

<http://hostProvider.org/FHIR#Patient.name>

CCDA:HumanName_e31bbafd-165c-4a88-8f8c-

684e2e5cafe1 .
<http://hostProvider.org/CCDA>

| | |
|---|---|
| a | owl:Ontology ; |

owl:imports
<http://hostProvider.org/HostProviderFHIR> ;

| | |
|---|---|
| owl:versionInfo | “Auto Generated from Profile” . |

CCDA:SectionComponent_5a90c841-125e-4c83-84ab-bb6275df4987

| | |
|---|---|
| a | <http://hostProvider.org/FHIR#SectionComponent> ; |

<http://hostProvider.org/FHIR#SectionComponent.code>

CCDA:CodeableConcept_11acfede-4fa2-4f6e-87e8-

38526a4a7645 ;
<http://hostProvider.org/FHIR#SectionComponent.entry>

CCDA:List_ef1b12ea-5764-49d8-998c-af9af9c9825d ;

<http://hostProvider.org/FHIR#SectionComponent.title>

“Vital Signs” .

CCDA:CodeableConcept_11acfede-4fa2-4f6e-87e8-38526a4a7645

| | |
|---|---|
| a | <http://hostProvider.org/FHIR#CodeableConcept> ; |

<http://hostProvider.org/FHIR#CodeableConcept.coding>

CCDA:Coding_38ca1604-9ddb-41d4-8239-76ca2f6abab1 .

CCDA:Coding_38ca1604-9ddb-41d4-8239-76ca2f6abab1

| | |
|---|---|
| a | HostProviderFHIR:Coding ; |

<http://hostProvider.org/FHIR#Coding.code>

“8716-3” .

CCDA:BundleEntryComponent_0c50e01c-d979-48fb-a94c-bf2801e5bb7b
a
<http://hostProvider.org/FHIR#BundleEntryComponent> ;
<http://hostProvider.org/FHIR#BundleEntryComponent.resource>

CCDA:Composition_8cfd1b14-0aac-478e-af60-

a234ace090da .
CCDA:ListEntryComponent_3386df8e-3610-4278-8e03-2b7f7b59178a

| | |
|---|---|
| a | <http://hostProvider.org/FHIR#ListEntryComponent> |

;
<http://hostProvider.org/FHIR#ListEntryComponent.item>

CCDA:Observation_1f20e739-e1db-49b3-ba0b-

3ff1982dd765 .
CCDA:List_ef1b12ea-5764-49d8-998c-af9af9c9825d

| | |
|---|---|
| a | <http://hostProvider.org/FHIR#List> ; |

<http://hostProvider.org/FHIR#List.entry>

CCDA:ListEntryComponent_3386df8e-3610-4278-8e03-

2b7f7b59178a ;
<http://hostProvider.org/FHIR#List.mode>

“working” ;

<http://hostProvider.org/FHIR#List.status>

“current” .

CCDA:HumanName_e31bbafd-165c-4a88-8f8c-684e2e5cafe1

| | |
|---|---|
| a | <http://hostProvider.org/FHIR#HumanName> ; |

<http://hostProvider.org/FHIR#HumanName.family>

“Coffin” ;

<http://hostProvider.org/FHIR#HumanName.given>

“Paulina ” .

CCDA:Bundle_c0e548fd-2145-4ef4-a227-286d4be52a5f

| | |
|---|---|
| a | <http://hostProvider.org/FHIR#Bundle> ; |

<http://hostProvider.org/FHIR#Bundle.entry>

CCDA:BundleEntryComponent_0c50e01c-d979-48fb-a94c-

bf2801e5bb7b .
CCDA:Composition_8cfd1b14-0aac-478e-af60-a234ace090da

| | |
|---|---|
| a | <http://hostProvider.org/FHIR#Composition> ; |

<http://hostProvider.org/FHIR#Composition.section>

CCDA:SectionComponent_5a90c841-125e-4c83-84ab-

bb6275df4987 ;
<http://hostProvider.org/FHIR#Composition.subject>

CCDA:Patient_758af88b-d467-45f4-9907-56896e5480f4

.
...

The TTL code portion example shown above is illustrative in nature, and is not to be considered limiting. It is contemplated herein that other types of modeling and model representation may be used, as would be understood by a person of skill in the relevant art(s) having the benefit of this disclosure. In embodiments, RDF ontology models may be implemented according to the “OWL” Web Ontology Language (see https://www.w3.org/TR/owl-ref/), and/or their equivalents.

As noted, inquiry classifier and response systems, along with further example embodiments thereof as described herein, are configured to automatically generate an ontology model (e.g., through RDF) of a FHIR model instance based on information in a CCDA document(s) and a standard RDF FHIR model using mappings therebetween. The standard RDF FHIR model is a uniform model for the FHIR standard and the instance model may include one or more of any element of the standard RDF FHIR model based on the presence of one or more information-value pairs in the CCDA document that correspond to the FHIR element.

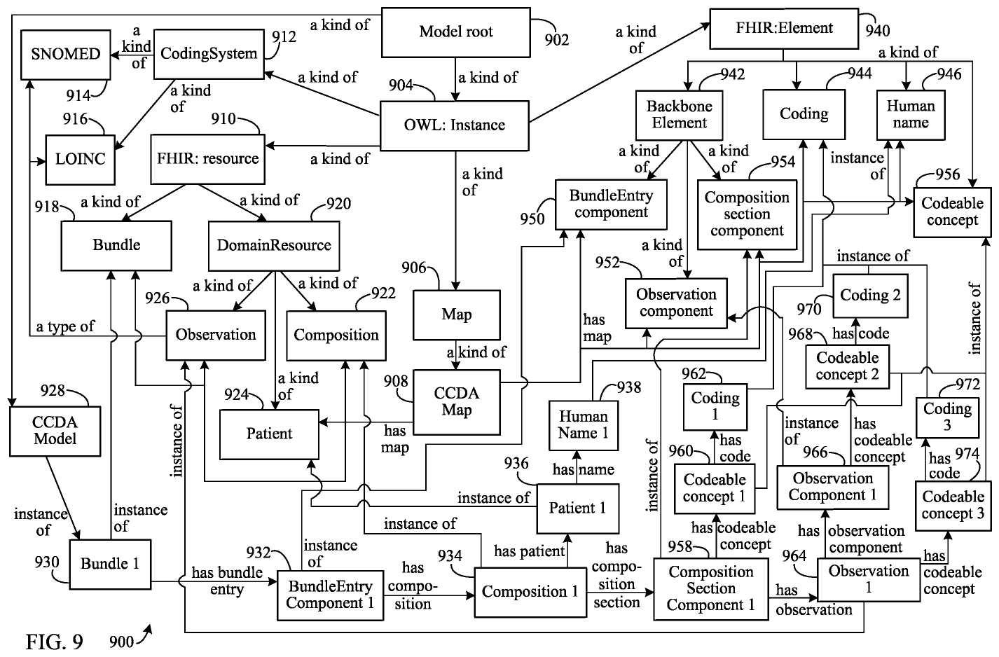

In FIG. 9, an ontology model 900 is shown, according to an embodiment. Ontology model 900 is a non-limiting, example ontology model embodiment that is illustrative in the context of the Example CCDA Document described above. In embodiments, ontology model 900 may correspond to an FHIR instance model representative of the Example CCDA Document.

Ontology model 900 includes a hierarchical structure of dependencies and attributes based on a root instance model root 902. Ontology model 900 is illustrated with respect to OWL according to an embodiment. From model root 902, an OWL: Instance 904 depends, which has “a kind of” attribute of model root 902. Ontology model 900 also includes a CCDA model 928 that depends from model root 902 and has “a kind of” attribute of model root 902.

From OWL: Instance 904, a Map element 906, a FHIR: resource element 910, a CodingSystem element 912, and a FHIR: Element 940 depend, each of which are “a kind of” OWL: Instance 904. A Systematized Nomenclature of Medicine (SNOMED) element 914 and a LOINC element 916 depend from CodingSystem element 912 and are “a kind of” CodingSystem element 912. From FHIR: resource element 910 a Bundle element 918 and a DomainResource 920 depend and are “a kind of” FHIR: resource element 910. A Bundle 1 930 is “an instance of” both CCDA Model 928 and Bundle element 918, and “has bundle entry” BundleEntry Component 1 932. From Map element 906, a CCDA Map element 908 depends that is “a kind of” Map element 906. Bundle element 918 includes a “has map” attribute of CCDA Map element 908.

An Observation element 926, a Composition element 922, and a Patient element 924 each depend from, and are “a kind of,” DomainResource 920. Observation element 926, Composition element 922, and Patient element 924 each include a “has map” attribute of CCDA Map element 908, and Observation element 926 is “a type of” SNOMED element 914 and/or LOINC element 916.

From FHIR: Element 940, a Backbone element 942, a Coding element 944, a Human name element 946, and a Codeable concept element 956 depend, each of which are “a kind of” FHIR: Element 940. Coding element 944, Human name element 946, and Codeable concept element 956 each include a “has map” attribute of CCDA Map element 908. A BundleEntry Component element 950, an Observation component element 952, and a Composition section component element 954 depend from, and are each “a kind of,” Backbone element 942, and each include a “has map” attribute of CCDA Map element 908.

BundleEntry Component 1 932 is an “instance of” BundleEntry component 950 and “has composition” Composition 1 934 which is an “instance of” Composition 1 element 922. Composition 1 element 922 “has patient” Patient 1 element 936 which is an “instance of” Patient element 924 and “has name” Human name 1 element 938. Human name 1 element 938 is an “instance of” Human name element 946. Composition 1 element 922 “has composition section” Composition section component 1 element 958 which is an “instance of” Composition section component element 954, “has codeable concept” Codeable concept 1 element 960, and “has observation” Observation 1 element 964. Codeable concept 1 element 960 “has code” Coding 1 element 962 which is an “instance of” Codeable concept element 956. Observation 1 element 964 is an “instance of” Observation element 926, “has observation component Observation component 1 element 966, and “has codeable concept” Codeable concept 3 element 974. Observation component 1 element 966 is an “instance of” Observation component element 952 and “has codeable concept” Codeable concept 2 element 968. Codeable concept 2 element 968 is an “instance of” Codeable concept element 956 and “has code” Coding 2 element 970 which is an “instance of” Coding element 944. Codeable concept 3 element 974 is an “instance of” Codeable concept element 956 and “has code” Coding 3 element 972 which is an “instance of” Coding element 944.

In ontology model 900, with respect to the Example CCDA Document described herein and the body weight observation of a patient, the patient's Human name 1 element may corresponds to “Paulina Coffin,” Composition section component 1 element 958 may correspond to “vital signs,” and Observation 1 element 964 may correspond to the measured body weight.

### IV. Further Example Embodiments and Advantages

As noted above, systems and devices, including inquiry classifier and response systems, may be configured in various ways to automatically generate FHIR resources such as FHIR ontology model instances from CCDA documents. It is also contemplated herein that generation of resources according to the FHIR standard may be performed based on non-CCDA documents, that generation of resources according to non-FHIR standards may be performed based on CCDA documents, and that generation of other non-FHIR standard formats may be performed based on non-CCDA documents. The FHIR ontology model instances, specific to a patient, are utilized in conjunction with ML models to determine answers to medical inquiries provided in text question data.

The described techniques and embodiments provide for the ability to automatically generate specifically tailored FHIR resources for the information in a CCDA document for use cases where only a section, or set, of specific entries from a CCDA document are needed for that use case. For example, automatic generation of specifically tailored FHIR resources for the information in a CCDA document allows for FHIR resources/resource bundles that can be stored in any compliant FHIR repository, with support for responding to FHIR queries, to respond to requests for specific components of CCDA documents with the appropriate set of FHIR resources that answers the query as directed to particular medical inquiries.

The systems and devices herein are configured to consume CCDA documents and generate the document's contents into a general ontological representation using RDF-specified mappings. This representation is then utilized to automatically generate a set of standard FHIR resources that can be stored in a standard FHIR repository, which may provide alternative data sources acquire information for fulfilling medical inquiries.

The described embodiments and techniques can also be used to generate FHIR resources and bundles equivalent to CCDA documents, allowing for the sharing of information to a healthcare entity that can consume FHIR resources and bundles, but not CCDA documents. By placing the generated FHIR resources in a FHIR repository, the described embodiments and techniques can also provide access to component information contained in a CCDA document via query, in a well-defined and structured manner.

Additionally, the ease of use for libraries used in implementations of document formatting standards is increased. For example, a specific instance of a FHIR model that corresponds to any information, or any requested information, in a CCDA document may be generated using a standard FHIR RDF model. That is, the generation of FHIR model instances is itself a model-based generation predicated on a standard FHIR RDF model. Thus the described embodiments and techniques provide for a flexible yet robust way to automatically generate a specific instance of a FHIR model that corresponds to any information, or any requested information, in a CCDA document. Such FHIR models are specifically tailored to provide, via queries, information required for particular medical inquiries, including as a microservice that supplements information for other applications, processes, and services hosted by a system.

The described techniques and embodiments may be utilized as or in any computing device or distributed computer system. The described techniques and embodiments provide value and efficiency benefits for large, and still increasing, networks of hosts, health care providers, and trading partners that desire to exchange clinical information, even those that are not capable of consuming CCDA documents.

The embodiments and techniques disclosed herein provide for a specific arrangement of components for automatically generating FHIR ontology model instances and FHIR resources, as well as utilizing such model instances and resources in conjunction with ML models and techniques for answering inquiries about patients. That is, the embodiments and techniques disclosed herein relate to a non-conventional and non-generic arrangement of elements in the FHIR model generation process, e.g., the model-based generation of each specific FHIR instance model based on a generated, standard FHIR model), with customized, user-specified elements specific to each use case that enables intelligent and efficient determinations of information sought.

The embodiments and techniques disclosed herein also provide for improving the technological process of computer-generated FHIR instance models and FHIR resources through the use of specific relationships and mappings that govern the generation of FHIR instance models and FHIR resources based on a generated standard model, rather than human-based implementations that simply involve the use of a computer, to determine instances for any number of sets of any given type of clinical observation. The described embodiments and techniques utilize specific relationships and mappings that allow for the generation of specific FHIR instance models and FHIR resources based on a generated standard model, and such a technique enables the automation of generating specific FHIR instance models and FHIR resources that previously could not be automated in such a manner. That is, human-based approaches do not involve generating an entire standard FHIR model upon which specific instance models that correspond to individual CCDA documents, or subsets of information therein, are generated. Accordingly, the efficiency of computer-based generation of FHIR instance models and FHIR resources is improved as is the generation of patient data payloads for responding to information requests.

Answerability ML models, e.g., BERT models, as described herein may be modified via training on parameters such as medical inquiries that are designated as answerable, or not, and associated answers determined by a medical professional for classification of later-received medical inquiries by the system, e.g., specifically trained for answerability of medical inquiries via training questions paired with answer data augmented by a medical professional, to determine if a given inquiry can be answered by the system.

Moreover, according to the described embodiments and techniques, any components of inquiry classifier and response systems and their functions may be caused to be activated for operation/performance thereof based on other operations, functions, actions, and/or the like, including initialization, completion, and/or performance of the, functions, actions, and/or the like.

In some example embodiments, one or more of the operations of the flowcharts described herein may not be performed. Moreover, operations in addition to or in lieu of the operations of the flowcharts described herein may be performed. Further, in some example embodiments, one or more of the operations of the flowcharts described herein may be performed out of order, in an alternate sequence, or partially (or completely) concurrently with each other or with other operations.

The further example embodiments and advantages described in this Section may be applicable to any embodiments disclosed in this Section or in any other Section of this disclosure.

Embodiments and techniques, including methods, described herein may be performed in various ways such as, but not limited to, being implemented by hardware, or hardware combined with one or both of software and firmware.

### V. Example Processing Device Implementations

Inquiry classifier and response system and device embodiments described herein, such as inquiry classifier and response system 104 of FIG. 1, inquiry classifier and response system 204 of FIG. 2, inquiry classifier and response system 300 of FIG. 3, and/or inquiry classifier and response system 556 of system 500 in FIG. 5, along with any respective components/subcomponents and/or further embodiments thereof, and/or any flowcharts, execution flows, further systems, sub-systems, and/or components, including other network-connected devices, disclosed herein may be implemented in hardware (e.g., hardware logic/electrical circuitry), or any combination of hardware with one or both of software (computer program code or instructions configured to be executed in one or more processors or processing devices) and firmware. In embodiments with respect to the example computer implementations in this Section, main memory, memory cards and memory sticks, memory devices, and/or the like may include and or implement the described techniques and embodiments.

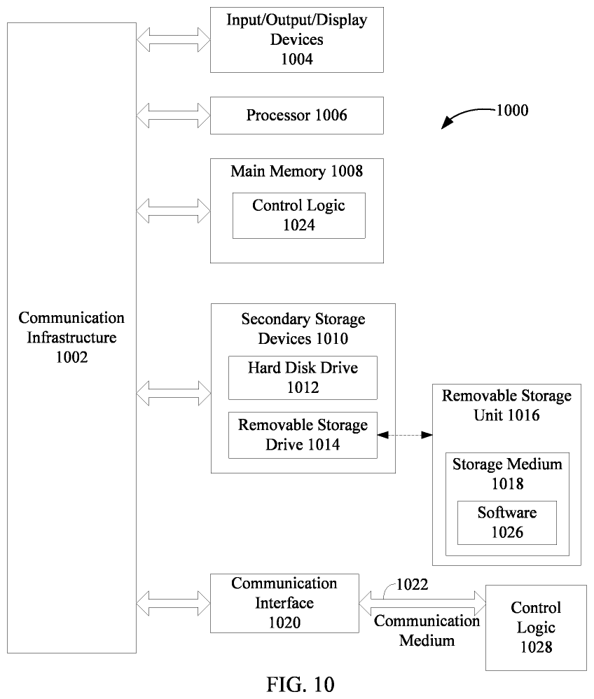

The embodiments described herein, including devices, systems, methods/processes, and/or apparatuses, may be implemented in or using processing devices, communication systems, servers, and/or, computers, such as a processing device 1000 shown in FIG. 10. It should be noted that processing device 1000 may represent mobile devices, communication devices/systems, entertainment systems/devices, processing devices, and/or traditional computers in one or more embodiments. For example, a system as described herein, and any of the sub-systems and/or components respectively contained therein and/or associated therewith, along with further embodiments thereof, may be implemented in or using one or more processing devices 1000 and/or similar computing devices.

Processing device 1000 can be any commercially available and well known communication device, processing device, and/or computer capable of performing the functions described herein, such as devices/computers available from International Business Machines®, Apple®, Sun®, HP®, Dell®, Cray®, Samsung®, Nokia®, etc. Processing device 1000 may be any type of computer, including a desktop computer, a server, etc., and may be a computing device or system within another device or system.

Processing device 1000 includes one or more processors (also called central processing units, or CPUs), such as a processor 1006. Processor 1006 is connected to a communication infrastructure 1002, such as a communication bus. In some embodiments, processor 1006 can simultaneously operate multiple computing threads, and in some embodiments, processor 1006 may comprise one or more processors.

Processing device 1000 also includes a primary or main memory 1008, such as random access memory (RAM). Main memory 1008 has stored therein control logic 1024 (computer software), and data.

Processing device 1000 also includes one or more secondary storage devices 1010. Secondary storage devices 1010 include, for example, a hard disk drive 1012 and/or a removable storage device or drive 1014, as well as other types of storage devices, such as memory cards and memory sticks. For instance, processing device 1000 may include an industry standard interface, such a universal serial bus (USB) interface for interfacing with devices such as a memory stick. Removable storage drive 1014 represents a floppy disk drive, a magnetic tape drive, a compact disk drive, an optical storage device, tape backup, etc.

Removable storage drive 1014 interacts with a removable storage unit 1016. Removable storage unit 1016 includes a computer useable or readable storage medium 1018 having stored therein computer software 1026 (control logic) and/or data. Removable storage unit 1016 represents a floppy disk, magnetic tape, compact disk, DVD, optical storage disk, or any other computer data storage device. Removable storage drive 1014 reads from and/or writes to removable storage unit 1016 in a well-known manner.

Processing device 1000 also includes input/output/display devices 1004, such as touchscreens, LED and LCD displays, monitors, keyboards, pointing devices, etc.

Processing device 1000 further includes a communication or network interface 1020. Communication interface 1020 enables processing device 1000 to communicate with remote devices. For example, communication interface 1020 allows processing device 1000 to communicate over communication networks or mediums 1022 (representing a form of a computer useable or readable medium), such as LANs, WANs, the Internet, etc. Network interface 1020 may interface with remote sites or networks via wired or wireless connections.

Control logic 1028 may be transmitted to and from processing device 1000 via the communication medium 1022.

Any apparatus or manufacture comprising a computer useable or readable medium having control logic (software) stored therein is referred to herein as a computer program product or program storage device. This includes, but is not limited to, processing device 1000, main memory 1008, secondary storage devices 1010, and removable storage unit 1016. Such computer program products, having control logic stored therein that, when executed by one or more data processing devices, cause such data processing devices to operate as described herein, represent embodiments.

Techniques, including methods, and embodiments described herein may be implemented by hardware (digital and/or analog) or a combination of hardware with one or both of software and/or firmware. Techniques described herein may be implemented by one or more components. Embodiments may comprise computer program products comprising logic (e.g., in the form of program code or software as well as firmware) stored on any computer useable medium, which may be integrated in or separate from other components. Such program code, when executed by one or more processor circuits, causes a device to operate as described herein. Devices in which embodiments may be implemented may include storage, such as storage drives, memory devices, and further types of physical hardware computer-readable storage media. Examples of such computer-readable storage media include, a hard disk, a removable magnetic disk, a removable optical disk, flash memory cards, digital video disks, random access memories (RAMs), read only memories (ROM), and other types of physical hardware storage media. In greater detail, examples of such computer-readable storage media include, but are not limited to, a hard disk associated with a hard disk drive, a removable magnetic disk, a removable optical disk (e.g., CDROMs, DVDs, etc.), zip disks, tapes, magnetic storage devices, MEMS (micro-electromechanical systems) storage, nanotechnology-based storage devices, flash memory cards, digital video discs, RAM devices, ROM devices, and further types of physical hardware storage media. Such computer-readable storage media may, for example, store computer program logic, e.g., program modules, comprising computer executable instructions that, when executed by one or more processor circuits, provide and/or maintain one or more aspects of functionality described herein with reference to the figures, as well as any and all components, capabilities, and functions therein and/or further embodiments described herein.

Such computer-readable media and/or computer-readable storage media (e.g., a computer-readable storage medium, or other derivative thereof) are distinguished from and non-overlapping with communication media and propagating signals (do not include communication media and propagating signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media that are separate and non-overlapping with embodiments directed to computer-readable storage media.

The techniques and embodiments described herein may be implemented as, or in, various types of circuits, devices, apparatuses, and systems. For instance, embodiments may be included, without limitation, in processing devices (e.g., illustrated in FIG. 10) such as computers and servers, as well as communication systems such as switches, routers, gateways, and/or the like, communication devices such as smart phones, home electronics, gaming consoles, entertainment devices/systems, etc. A device, as defined herein, is a machine or manufacture as defined by 35 U.S.C. § 101. That is, as used herein, the term “device” refers to a machine or other tangible, manufactured object and excludes software and signals. Devices may include digital circuits, analog circuits, or a combination thereof. Devices may include one or more processor circuits (e.g., central processing units (CPUs), processor 1006 of FIG. 10), microprocessors, digital signal processors (DSPs), and further types of physical hardware processor circuits) and/or may be implemented with any semiconductor technology in a semiconductor material, including one or more of a Bipolar Junction Transistor (BJT), a heterojunction bipolar transistor (HBT), a metal oxide field effect transistor (MOSFET) device, a metal semiconductor field effect transistor (MESFET) or other transconductor or transistor technology device. Such devices may use the same or alternative configurations other than the configuration illustrated in embodiments presented herein.

### VI. Conclusion

While various embodiments have been described above, it should be understood that they have been presented by way of example only, and not limitation. It will be apparent to persons skilled in the relevant art that various changes in form and detail can be made therein without departing from the spirit and scope of the embodiments. Thus, the breadth and scope of the embodiments should not be limited by any of the above-described exemplary embodiments, but should be defined only in accordance with the following claims and their equivalents.

What is claimed is:

1. A system, comprising:
a memory configured to store program instructions; and
at least one processor configured to execute the program instructions that cause the at least one processor to:
receive, from a requestor device and via an external network, a document that includes clinical data of a patient and text question data that includes a set of medical inquiries;
generate an ontology model instance that includes objects representative of pairs comprising information identifiers and corresponding value strings from the clinical data in the document;
determine a question category of a plurality of question categories to which the text question data corresponds via a first machine learning model;
map portions of the text question data to one or more of the objects via a second machine learning model and based at least on the question category;
query the ontology model instance, utilizing one or more of the information identifiers of the one or more of the objects as query terms, and generate a query result that includes respectively corresponding value strings of the one or more of the objects to which the portions were mapped; and
transmit data associated with the patient, to the requestor device and via the external network, based on the query result as responsive to the text question data.

2. The system of claim 1, wherein the one or more of the information identifiers causes said query the ontology model instance to query less than all objects in the ontology model instance based on said map the portions of the text question data to the one or more of the objects.

3. The system of claim 1, wherein the text question data comprises natural language text;
wherein execution of the program instructions further cause the at least one processor to:
clean the natural language text by a natural language processor prior to said determine the question category.

4. The system of claim 1, wherein execution of the program instructions further cause the at least one processor to perform at least one of to:
generate a confidence index of the text question data, indicative of a medical inquiry of the set being answerable, via a third machine learning model prior to the question category being determined;
or
determine, via the second machine learning model and prior to said transmit the data associated with the patient, that the query result includes a result portion, corresponding to at least one of the set of medical inquiries, which includes a value string that is different from an answer to the at least one of the set of medical inquiries, and
generate new data based on the query result as at least a portion of the data associated with the patient.

5. The system of claim 1, wherein the document is in a CCDA (Consolidated Clinical Document Architecture) format and the ontology model instance is in a FHIR (Fast Healthcare Interoperability Resources) format.

6. The system of claim 1, wherein execution of the program instructions further cause the at least one processor to:
determine one of the set of medical inquiries for which any corresponding question category of the plurality of question categories is not determined via the first machine learning model;
identify an alternate data source by the first machine learning model that includes additional data that is associated with the one of the set of medical inquiries; and
transmit the additional data as at least a portion of the data associated with the patient.

7. The system of claim 1, wherein execution of the program instructions further cause the at least one processor to:
retrain at least one of the first machine learning model or the second machine learning model based at least on one or more of the set of medical inquiries or cleaned natural language text associated therewith; and
deploy the at least one of the first machine learning model or the second machine learning model, subsequent to said retrain, against another document that is subsequently received and that includes clinical data of another patient and other text question data that includes another set of medical inquiries for improved processing thereof.

8. A method, implemented by a computing system, comprising:
receiving, from a requestor device and via an external network, a document that includes clinical data of a patient and text question data that includes a set of medical inquiries;
generating an ontology model instance that includes objects representative of pairs comprising information identifiers and corresponding value strings from the clinical data in the document;
determining a question category of a plurality of question categories to which the text question data corresponds via a first machine learning model;
mapping portions of the text question data to one or more of the objects via a second machine learning model and based at least on the question category;
querying the ontology model instance, utilizing one or more of the information identifiers of the one or more of the objects as query terms, and generate a query result that includes respectively corresponding value strings of the one or more of the objects to which the portions were mapped; and
transmitting data associated with the patient, to the requestor device and via the external network, based on the query result as responsive to the text question data.

9. The method of claim 8, wherein, via on the one or more of the information identifiers, said querying the ontology model instance to queries less than all objects in the ontology model instance based on said map the portions of the text question data to the one or more of the objects.

10. The method of claim 8, wherein the text question data comprises natural language text; and
wherein the method further comprises:
cleaning the natural language text by a natural language processor prior to said determining the question category.

11. The method of claim 8, further comprising at least one of:
generating a confidence index of the text question data, indicative of a medical inquiry of the set being answerable, via a third machine learning model prior to the question category being determined;
or
determining, via the second machine learning model and prior to said transmit the data associated with the patient, that the query result includes a result portion, corresponding to at least one of the set of medical inquiries, which includes a value string that is different from an answer to the at least one of the set of medical inquiries, and
generating new data based on the query result as at least a portion of the data associated with the patient.

12. The method of claim 8, wherein the document is in a CCDA (Consolidated Clinical Document Architecture) format and the ontology model instance is in a FHIR (Fast Healthcare Interoperability Resources) format.

13. The method of claim 8, further comprising:
determining one of the set of medical inquiries for which any corresponding question category of the plurality of question categories is not determined via the first machine learning model;
identifying an alternate data source by the first machine learning model that includes additional data that is associated with the one of the set of medical inquiries; and
transmitting the additional data as at least a portion of the data associated with the patient.

14. The method of claim 8, further comprising:
retraining at least one of the first machine learning model or the second machine learning model based at least on one or more of the set of medical inquiries or cleaned natural language text associated therewith; and
deploying the at least one of the first machine learning model or the second machine learning model, subsequent to said retrain, against another document that is subsequently received and that includes clinical data of another patient and other text question data that includes another set of medical inquiries for improved processing thereof.

15. A computer-readable storage medium having program instructions encoded thereon that, when executed by one or more processors, performs a computer-implemented method, the method comprising:
receiving, from a requestor device and via an external network, a document that includes clinical data of a patient in a CCDA (Consolidated Clinical Document Architecture) format and text question data that includes a set of medical inquiries;
generating an ontology model instance in a FHIR (Fast Healthcare Interoperability Resources) format that includes objects representative of pairs comprising information identifiers and corresponding value strings from the clinical data in the document;
determining a question category of a plurality of question categories to which the text question data corresponds via a first machine learning model;
mapping portions of the text question data to one or more of the objects via a second machine learning model and based at least on the question category;
querying the ontology model instance, utilizing one or more of the information identifiers of the one or more of the objects as query terms, and generate a query result that includes respectively corresponding value strings of the one or more of the objects to which the portions were mapped; and
transmitting data associated with the patient, to the requestor device and via the external network, based on the query result as responsive to the text question data.

16. The computer-readable storage medium of claim 15, wherein, via on the one or more of the information identifiers, said querying the ontology model instance to queries less than all objects in the ontology model instance based on said map the portions of the text question data to the one or more of the objects.

17. The computer-readable storage medium of claim 15, wherein the text question data comprises natural language text; and
wherein the method further comprises:
cleaning the natural language text by a natural language processor prior to said determining the question category.

18. The computer-readable storage medium of claim 15, wherein the method further comprises at least one of:
generating a confidence index of the text question data, indicative of a medical inquiry of the set being answerable, via a third machine learning model prior to the question category being determined;
or
determining, via the second machine learning model and prior to said transmit the data associated with the patient, that the query result includes a result portion, corresponding to at least one of the set of medical inquiries, which includes a value string that is different from an answer to the at least one of the set of medical inquiries, and
generating new data based on the query result as at least a portion of the data associated with the patient.

19. The computer-readable storage medium of claim 15, wherein the method further comprises:
determining one of the set of medical inquiries for which any corresponding question category of the plurality of question categories is not determined via the first machine learning model;
identifying an alternate data source by the first machine learning model that includes additional data that is associated with the one of the set of medical inquiries; and
transmitting the additional data as at least a portion of the data associated with the patient.

20. The computer-readable storage medium of claim 15, wherein the method further comprises:
retraining at least one of the first machine learning model or the second machine learning model based at least on one or more of the set of medical inquiries or cleaned natural language text associated therewith; and
deploying the at least one of the first machine learning model or the second machine learning model, subsequent to said retrain, against another document that is subsequently received and that includes clinical data of another patient and other text question data that includes another set of medical inquiries for improved processing thereof.
